(19) United States
(12) Patent Application Publication
(10) Pub. No.: US 2008/0116096 A1
(43) Pub. Date: May 22, 2008

(54) Liquid-permeable appliance for delivering a composition

(76) Inventors: Kroy D. Johnson, Menasha, WI (US); Wael R. Joseph, Appleton, WI (US); Kenneth B. Close, New London, WI (US); Jonathan K. Arendt, Appleton, WI (US)

Correspondence Address: KIMBERLY-CLARK WORLDWIDE, INC.;Catherine E. Wolf, 401 NORTH LAKE STREET, NEENAH, WI, 54956, US

(21) Appl. No.: 11/601,033

(22) Filed: Nov. 17, 2006

(51) Int. Cl. B65D 85/18; A61F 13/84

(52) U.S. Cl. 206/459.5; 604/385.01; 206/525

(57) ABSTRACT

An appliance, such as a sleeve, sock, glove, or patch, that comprises a liquid-permeable substrate and a substantially dry and/or immobile composition (i.e., the formulation is substantially free of water so that the formulation is a solid or semi-solid formulation at temperatures typifying transport, storage, and/or use conditions), effectively and comfortably treats the skin or tissue of a user. Humectants, materials of an occlusive nature, and numerous other ingredients may be included in the formulation. The appliance can comprise elastomeric materials that facilitate contact between the appliance and skin.

### BACKGROUND

People rely on various kinds of formulations or compositions for health and/or hygiene benefits.

One type of health-and-hygiene formulation is used to moisturize or hydrate skin. Generally, two categories of ingredients are used when moisturizing and/or hydrating skin. Humectants are used to transport moisture from the environment (primarily water vapor in air), or from deeper layers of the skin, to the skin surface (where skin typically can become dry). One example of a humectant is glycerin. Such materials tend to be hydrophilic (i.e., water loving), and are generally non-greasy, light in weight, and quick to apply. Occlusive materials are used to trap moisture already present in the skin. These materials tend to create a water barrier over the skin, thereby inhibiting the transport of water from the skin to the external environment (i.e., transepidermal water loss, or “TEWL”). An example of an occlusive material is petrolatum. Such materials tend to be heavier, effective over longer periods of time, and often contain oily and/or greasy ingredients.

Often people apply such formulations directly to skin using their hands. After application of the formulation to the body, any excess formulation remaining on the hands typically needs to be removed—a potential inconvenience to the user of the formulation.

In some instances, gloves, socks, sleeves, or other appliances have been used in conjunction with formulations. For example, a user either applies a formulation to his or her hand or foot, and then dons or applies a glove over the treated hand or a sock over the treated foot. Alternatively, a user slips on a glove or sock that has been pre-treated with a skin-care formulation. Unfortunately, such items have typically been made of a polymeric material (e.g., neoprene rubber) that does not conform readily to surfaces and contours of, for example, a foot or hand. Furthermore, these items typically lack a cloth-like appearance and feel.

If the formulation contains significant amounts of water, then the appliance will generally be adapted to contain said water. For example, an appliance made solely of a relatively open and porous web of cellulosic fibers would likely readily absorb some portion of the water (untreated cellulose, having polar hydroxyl groups, is generally regarded as hydrophilic, or “water-loving”). The absorbed water and any dissolved materials could migrate to the exterior surface of the appliance, potentially contaminating any surface touched or contacted by a wearer of the appliance. Furthermore, if water migrating through the appliance evaporated, then the formulation may dry out more rapidly, thereby reducing the time interval in which a user benefits from a moisturizing effect on the skin.

One approach to addressing issues like that described in the previous paragraph is to make an appliance that has a liquid- or water-impermeable layer that inhibits or reduces passage of water through the appliance. Alternatively, an appliance comprising a liquid- or water-permeable layer could be employed with a formulation selected to be substantially dry (i.e., the formulation employs small amounts of water, if any).

What is needed is an appliance that promotes health and/or hygiene by facilitating application of a formulation or composition to skin or tissue (i.e., the appliance delivers a composition so that the composition contacts skin or tissue of a user of the appliance), and which: has a liquid-permeable substrate that may be adapted to conform readily to the contours and surfaces of parts of the body to which the appliance is applied (e.g., by employing biaxially elastomeric materials, such as a porous elastomeric composite—including, for example, spaced-apart, substantially parallel elastic strands attached to meltblown fiber—sandwiched between two fibrous webs; and a formulation that is substantially dry (i.e., is substantially free of water such that most or all of the formulation remains in, or on the body-facing side of, the appliance, rather than migrating to the exterior of the appliance, where the formulation might then transfer to surfaces other than the user's skin—typically this means that the formulation is a solid or semi-solid, or substantially immobile); wherein the appliance is typically adapted to be used a single time and then disposed of. In some cases the liquid-permeable substrate is a fibrous material, such as a non-woven. In other cases the liquid-permeable substrate is a porous or apertured film. Other layers may be present (e.g., a porous film may be attached to, for example, one or more fibrous layers). Or, as noted above, a liquid-permeable substrate may comprise an elastomeric composite attached to one or more additional layers (such as one or more fibrous layers). Other configurations are possible, so long as the substrate, whether made from one layer or a plurality of layers, is liquid permeable.

### SUMMARY

We have determined that an appliance, such as a sleeve, sock, or glove, that comprises a liquid-permeable substrate and a substantially dry composition (i.e., the formulation is sufficiently free of water so that the formulation is a solid or semi-solid formulation at temperatures typifying use, storage, and/or transport conditions), effectively and comfortably treats the skin or tissue of a user. Humectants, materials of an occlusive nature, and numerous other ingredients may be included in the formulation, examples of which are provided below in the Description section.

Typically the formulation includes a material such as polyethylene glycol, wax, anhydrous petrolatum, or some other polymeric material of sufficient molecular weight that the formulation is a solid or semi-solid, i.e., the formulation is sufficiently viscous or solid such that it does not migrate through the liquid-permeable substrate from the time of manufacture through the time of use. Liquid ingredients may be employed when making the formulation, so long as the ingredients are immobilized in the final formulation (i.e., the formulation, as stated above, is a solid or semi-solid such that it does not migrate through the liquid-permeable substrate from the time of manufacture through the time of use).

In one embodiment of the present invention, the liquid-permeable substrate comprises an apertured or porous film. The film may be attached to other liquid-permeable layer(s), such as an outer fibrous substrate. This outer substrate can impart a cloth-like appearance and feel to the appliance. In one version of the present invention, the outer fibrous substrate is attached to the film at discrete points or regions (as with, e.g., thermal point bonding). In another version of the present invention, the film is in a stretched condition when the outer fibrous substrate is bonded to it (e.g., at discrete points or regions), and then allowed to retract, thereby helping effect an increased rugosity or increase in the number of undulations associated with the outer fibrous layer.

In another version of the invention, the liquid-permeable layer, in this case a porous or apertured film, is attached to an inner fibrous substrate. In one version of the present invention, the inner fibrous substrate is attached to the film at discrete points or regions (as with, e.g., thermal point bonding). In another version of the present invention, the film is in a stretched condition when the outer fibrous substrate is bonded to it (e.g., at discrete points or regions), and then allowed to retract, thereby helping effect an increased rugosity or increase in the number of undulations associated with the inner fibrous layer.

In another version of the invention, the porous or apertured film is attached to both an inner and outer fibrous layer.

In another version of the present invention, the aforementioned film comprises an elastomeric polymer, thereby imparting elasticity to the appliance as a whole. As noted above, a film—in this case one employing an elastomeric material, or materials—may be attached to an inner and/or outer fibrous layer either uniformly, or at selected locations, including discrete points or regions (e.g., as result from thermal point bonding). Furthermore, a film employing elastomeric material(s) may be stretched prior to bonding or attachment to the outer fibrous layer, and then allowed to retract.

The film (or other material) can also be made to stretch, and recover some portion of its unstretched dimensions, by slitting or aperturing the film (or other material)—i.e., as an external force is applied, that portion of the film proximate to opposing sides of slits or apertures can separate, in effect allowing the slits or aperatures to open up—which allows the film as a whole to elongate; etc.

Note too that any attached fibrous layer may employ elastomeric materials and/or be formed in a way that the fibrous layer has some ability to stretch and then recover some portion of its unstretched dimensions.

In another version of the invention, the liquid-permeable layer is a fibrous structure. Typically the fibrous structure will be a nonwoven material that employs synthetic fibers formed by, e.g., some type of extrusion process.

The formulation associated with an appliance of the present invention is substantially dry (i.e., substantially free of water such that the formulation is a solid or semi-solid at the temperatures typical of use conditions). The formulation may employ any number of ingredients, such as emollients, humectants, and the like. Typically the water content of the formulation will be less than about 10% by weight of the formulation; suitably less than about 5% by weight of the formulation; and particularly less than 3% by weight of the formulation. Water contents at which the resulting formulation is not a solid or semi-solid—i.e., the resulting formulation is substantially a liquid such that the formulation may migrate through to the exterior portion of the liquid-permeable appliance during normal storage, transport, or use conditions—will not be employed with an appliance of the present invention.

These and other versions of the invention are described more fully below.

### DRAWINGS

FIG. 1A representatively illustrates one version of a substrate of the present invention.

FIG. 1B representatively illustrates one version of a substrate of the present invention.

FIG. 1C representatively illustrates one version of a substrate of the present invention.

FIG. 2 representatively illustrates one version of a substrate cut so that the substrate's perimeter defines the shape of a hand.

FIG. 2A representatively illustrates one version of an appliance of the present invention.

FIG. 3 representatively illustrates one version of a substrate cut so as to form a foot appliance of the present invention.

FIG. 3A representatively illustrates one version of an appliance of the present invention.

### DEFINITIONS

Within the context of this specification, each term or phrase below includes the following meaning or meanings:

“Attach” and its derivatives refer to the joining, adhering, connecting, bonding, sewing together, depositing on, associating with, or the like, of two elements. Two elements will be considered to be attached together when they are integral with one another or attached directly to one another or indirectly to one another, such as when each is directly attached to intermediate elements. “Attach” and its derivatives include permanent, releasable, or refastenable attachment. In addition, the attachment can be completed either during the manufacturing process or by the end user.

“Bond” and its derivatives refer to the joining, adhering, connecting, attaching, sewing together, or the like, of two elements. Two elements will be considered to be bonded together when they are bonded directly to one another or indirectly to one another, such as when each is directly bonded to intermediate elements. “Bond” and its derivatives include permanent, releasable, or refastenable bonding.

“Coform” refers to a blend of meltblown fibers and absorbent fibers such as cellulosic fibers that can be formed by air forming a meltblown polymer material while simultaneously blowing air-suspended fibers into the stream of meltblown fibers. The coform material may also include other materials, such as superabsorbent materials. The meltblown fibers and absorbent fibers are collected on a forming surface, such as provided by a foraminous belt. The forming surface may include a gas-pervious material that has been placed onto the forming surface.

“Composition,” “formulation,” or their derivatives, when used in the context of a material applied to, deposited on, or associated with an appliance of the present invention, refers to the various materials that help improve the health and/or hygiene of a user of the appliance, primarily by helping improve the health and/or hygiene of skin or tissue that contacts the material.

“Connect” and its derivatives refer to the joining, adhering, bonding, attaching, sewing together, or the like, of two elements. Two elements will be considered to be connected together when they are connected directly to one another or indirectly to one another, such as when each is directly connected to intermediate elements. “Connect” and its derivatives include permanent, releasable, or refastenable connection. In addition, the connecting can be completed either during the manufacturing process or by the end user.

“Disposable” refers to articles which are designed to be discarded after a limited use rather than being laundered or otherwise restored for reuse.

The terms “disposed on,” “disposed along,” “disposed with,” or “disposed toward” and variations thereof are intended to mean that one element can be integral with another element, or that one element can be a separate structure bonded to or placed with or placed near another element.

“Fiber” refers to a continuous or discontinuous member having a high ratio of length to diameter or width. Thus, a fiber may be a filament, a thread, a strand, a yarn, or any other member or combination of these members.

“Hydrophilic” describes materials or surfaces which are wetted by aqueous liquids in contact with the material or surface. The degree of wetting of the material or surface can, in turn, be described in terms of the contact angles and the surface tensions of the liquids and materials (or surfaces) involved.

“Layer” when used in the singular can have the dual meaning of a single element or a plurality of elements.

“Liquid impermeable,” when used in describing a layer or multi-layer laminate means that liquid, such as water, will not pass to any appreciable extent through the layer or laminate, under ordinary use conditions, in a direction generally perpendicular to the plane of the layer or laminate at the point of liquid contact.

“Liquid permeable” refers to any material that is not liquid impermeable.

“Meltblown” refers to fibers formed by extruding a molten thermoplastic material through a plurality of fine, usually circular, die capillaries as molten threads or filaments into converging high velocity gas (e.g., air) streams, generally heated, which attenuate the filaments of molten thermoplastic material to reduce their diameters. Thereafter, the meltblown fibers are carried by the high velocity gas stream and are deposited on a collecting surface to form a web of randomly dispersed meltblown fibers. Such a process is disclosed, for example, in U.S. Pat. No. 3,849,241 to Butin et al. Meltblowing processes can be used to make fibers of various dimensions, including macrofibers (with average diameters from about 40 to about 100 microns), textile-type fibers (with average diameters between about 10 and 40 microns), and microfibers (with average diameters less than about 10 microns). Meltblowing processes are particularly suited to making microfibers, including ultra-fine microfibers (with an average diameter of about 3 microns or less). A description of an exemplary process of making ultra-fine microfibers may be found in, for example, U.S. Pat. No. 5,213,881 to Timmons, et al. Meltblown fibers may be continuous or discontinuous and are generally self bonding when deposited onto a collecting surface.

“Member” when used in the singular can have the dual meaning of a single element or a plurality of elements.

“Nonwoven” and “nonwoven web” refer to materials and webs of material that are formed without the aid of a textile weaving or knitting process. For example, nonwoven materials, fabrics or webs have been formed from many processes such as, for example, meltblowing processes, spunbonding processes, air laying processes, and bonded carded web processes.

“Spunbonded fibers” refers to small diameter fibers which are formed by extruding molten thermoplastic material as filaments from a plurality of fine, usually circular capillaries of a spinneret with the diameter of the extruded filaments then being rapidly reduced to fibers as by, for example, in U.S. Pat. No. 4,340,563 to Appel et al., and U.S. Pat. No. 3,692,618 to Dorschner et al., U.S. Pat. No. 3,802,817 to Matsuki et al., U.S. Pat. Nos. 3,338,992 and 3,341,394 to Kinney, U.S. Pat. No. 3,502,763 to Hartman, and U.S. Pat. No. 3,542,615 to Dobo et al., the contents of which are incorporated herein by reference in their entirety. Spunbond fibers are generally continuous and have diameters generally greater than about 7 microns, more particularly, between about 10 and about 20 microns.

“Stretch bonded laminate” refers to a composite material having at least two layers in which one layer is a gatherable layer and the other layer is an elastic layer. The layers are joined together when the elastic layer is extended from its original condition so that upon relaxing the layers, the gatherable layer is gathered. Such a multilayer composite elastic material may be stretched to the extent that the nonelastic material gathered between the bond locations allows the elastic material to elongate. One type of stretch bonded laminate is disclosed, for example, by U.S. Pat. No. 4,720,415 to Vander Wielen et al., the content of which is incorporated herein by reference in its entirety. Other composite elastic materials are disclosed in U.S. Pat. No. 4,789,699 to Kieffer et al., U.S. Pat. No. 4,781,966 to Taylor and U.S. Pat. Nos. 4,657,802 and 4,652,487 to Morman and U.S. Pat. No. 4,655,760 to Morman et al., the contents of which are incorporated herein by reference in their entirety.

“Necking” or “neck stretching” interchangeably refer to a method of elongating a nonwoven fabric, generally in the machine direction, to reduce its width (cross-machine direction) in a controlled manner to a desired amount. The controlled stretching may take place under cool, room temperature or greater temperatures and is limited to an increase in overall dimension in the direction being stretched up to the elongation required to break the fabric, which in most cases is about 1.2 to 1.6 times. When relaxed, the web retracts toward, but does not return to, its original dimensions. Such a process is disclosed, for example, in U.S. Pat. No. 4,443,513 to Meitner and Notheis, U.S. Pat. Nos. 4,965,122, 4,981,747 and 5,114,781 to Morman and U.S. Pat. No. 5,244,482 to Hassenboehier Jr. et al., the contents of which are incorporated herein by reference in their entirety.

“Necked material” refers to any material which has undergone a necking or neck stretching process.

“Reversibly necked material” refers to a material that possesses stretch and recovery characteristics formed by necking a material, then heating the necked material, and cooling the material. Such a process is disclosed in U.S. Pat. No. 4,965,122 to Morman, commonly assigned to the assignee of the present invention, and incorporated by reference herein in its entirety. As used herein, the term “neck bonded laminate” refers to a composite material having at least two layers in which one layer is a necked, non-elastic layer and the other layer is an elastic layer. The layers are joined together when the non-elastic layer is in an extended (necked) condition. Examples of neck-bonded laminates are such as those described in U.S. Pat. Nos. 5,226,992, 4,981,747, 4,965,122 and 5,336,545 to Morman, the contents of which are incorporated herein by reference in their entirety.

“Stitchbonded” refers to a process in which materials (fibers, webs, films, etc.) are joined by stitches sewn or knitted through the materials. Examples of such processes are illustrated in U.S. Pat. No. 4,891,957 to Strack et al. and U.S. Pat. No. 4,631,933 to Carey, Jr., the contents of which are incorporated herein by reference in their entirety.

“Ultrasonic bonding” refers to a process in which materials (fibers, webs, films, etc.) are joined by passing the materials between a sonic horn and anvil roll. An example of such a process is illustrated in U.S. Pat. No. 4,374,888 to Bornslaeger, the content of which is incorporated herein by reference in its entirety.

“Thermal point bonding” involves passing materials (fibers, webs, films, etc.) to be bonded between a heated calender roll and an anvil roll. The calender roll is usually, though not always, patterned in some way so that the entire fabric is not bonded across its entire surface, and the anvil roll is usually flat. As a result, various patterns for calender rolls have been developed for functional as well as aesthetic reasons. Typically, the percent bonding area varies from around 10 percent to around 30 percent of the area of the fabric laminate. As is well known in the art, thermal point bonding holds the laminate layers together and imparts integrity to each individual layer by bonding filaments and/or fibers within each layer.

“Elastic” refers to any material, including a film, fiber, nonwoven web, or combination thereof, which upon application of a biasing force in at least one direction, is stretchable to a stretched, biased length which is at least about 110 percent, suitably at least about 130 percent, and particularly at least about 150 percent, its relaxed, unstretched length, and which will recover at least 15 percent of its elongation upon release of the stretching, biasing force. In the present application, a material need only possess these properties in at least one direction to be defined as elastic.

“Extensible and retractable” refers to the ability of a material to extend upon stretch and retract upon release. Extensible and retractable materials are those which, upon application of a biasing force, are stretchable to a stretched, biased length and which will recover a portion, suitably at least about 15 percent, of their elongation upon release of the stretching, biasing force.

As used herein, the terms “elastomer” or “elastomeric” refer to polymeric materials that have properties of stretchability and recovery.

“Stretch” refers to the ability of a material to extend upon application of a biasing force. Percent stretch is the difference between the initial dimension of a material and that same dimension after the material has been stretched or extended following the application of a biasing force. Percent stretch may be expressed as [(stretched length—initial sample length)/initial sample length]×100. For example, if a material having an initial length of one (1) inch is stretched 0.50 inch, that is, to an extended length of 1.50 inches, the material can be said to have a stretch of 50 percent.

“Recover” or “recovery” refers to a contraction of a stretched material upon termination of a biasing force following stretching of the material by application of the biasing force. For example, if a material having a relaxed, unbiased length of one (1) inch is elongated 50 percent by stretching to a length of one and one half (1.5) inches the material would have a stretched length that is 150 percent of its relaxed length. If this exemplary stretched material contracted, that is recovered to a length of one and one tenth (1.1) inches after release of the biasing and stretching force, the material would have recovered 80 percent (0.4 inch) of its elongation.

“Water impermeable,” when used in describing a layer or multi-layer laminate means that water or water vapor will not pass to any appreciable extent through the layer or laminate, under ordinary use conditions, in a direction generally perpendicular to the plane of the layer or laminate at the point of liquid contact.

“Water permeable” refers to any material that is not water impermeable.

These terms may be defined with additional language in the remaining portions of the specification.

### Description

Various problems associated with skin or tissue may diminish the health and/or hygiene of a person or animal. For example, dryness of the skin of the hands, feet, extremities, joints, or other parts of a body is a common problem, especially when skin is exposed to cold and/or dry conditions. This may be especially true for older individuals. Various formulations designed to moisturize skin are often used to address this problem. Some formulations require a substantial amount of time to deliver the anticipated benefit. For many currently available formulations, any resulting skin benefit may last a relatively short period of time after the formulation has been applied. The present invention characterizes an appliance for delivering a formulation or composition to tissue or skin.

One example of such a composition is a skin-care formulation for moisturizing skin. Such formulations typically work through at least one of two mechanisms: occlusivity or humectancy. Skin-care formulations relying on occlusivity form a relatively water-vapor-impermeable film on and/or in a skin surface. This occlusive film results in the accumulation of water underneath the film as the skin undergoes the natural process of trans-epidermal water loss. One advantage of the occlusivity approach includes the ability to provide a skin-moisturization benefit for an extended period of time. Occlusive ingredients are typically hydrophobic in nature and are generally not easily washable, which contribute to their ability to provide long-term moisturization of skin. Formulations with ingredients having occlusive properties (such as peterolatum, waxes, vegetable oils, mineral oil, etc.) are perceived by some as having undesirable aesthetic and/or feel attributes. Occlusive formulations may also lack the ability to provide quick moisturization as such formulations depend on the relatively slow process of water accumulation due to trans-epidermal water loss to deliver the moisturizing benefit.

Humectant formulations have the ability to attract water vapor (moisture) from the atmosphere and bring it to the skin surface, which results in increased skin hydration and alleviation of dryness. This process of attracting moisture is frequently referred to as “hygroscopicity”. Humectant formulations have the advantage of delivering a moisturization benefit and dryness relief within a short period of time. Humectant formulations are typically hydrophilic (as noted above, “water loving”) in nature and generally have the ability to attract a significant amount of water. Such formulations are generally perceived as having a light, pleasant feel (i.e., light in weight and/or viscosity) on the skin and typically are aesthetically preferred by the user (relative to skin-care compositions that function by occlusivity). Examples of humectant ingredients include glycerin, urea, sodium lactate, polysaccharides, and the like. Unlike occlusive formulations, humectant formulations generally lack the ability to provide moisturization over an extended period of time.

In one aspect of the present invention, a formulation having less than about 10% by weight water is applied to, or associated with, the interior or body-facing side of an appliance such as a glove, sock, or sleeve. The appliance comprises a liquid-permeable substrate, such as a porous or apertured film (with or without additional layers, such as an inner fibrous layer, an outer fibrous layer, or both); or a fibrous layer; or some other liquid-permeable material. Because the formulation employs a relatively small amount of water, it tends to have a higher viscosity (i.e., it tends to flow less; as stated above, the formulation will typically be a solid or semi-solid). While the appliance employs liquid-permeable material(s), the appliance is adapted to inhibit migration or transport of the formulation through the appliance to its exterior surface (where, as mentioned above, the formulation would be available to contact or contaminate surfaces with which a user of the appliance came into contact).

When the liquid-permeable substrate comprises an apertured or porous film, the film may comprise elastomeric materials. A suitable class of film materials includes a thermoplastic elastomeric polyolefin polymer, such as, for example, polypropylene. Suitable propylene polymers are commercially available under the designations VISTAMAXX™ from ExxonMobil Chemical Co. of Houston, Tex.; FINA™ (e.g., 8573) from Atofina Chemicals of Feluy, Belgium; TAFMER™ available from Mitsui Petrochemical Industries; and VERSIFY™ available from Dow Chemical Co. of Midland, Mich. Other examples of suitable propylene polymers are described in U.S. Pat. No. 7,105,609 to Datta, et al.; U.S. Pat. No. 6,500,563 to Datta, et al.; U.S. Pat. No. 5,539,056 to Yang, et al.; and U.S. Pat. No. 5,596,052 to Resconi, et al., which are incorporated herein in their entirety by reference thereto for all purposes.

Of course, other thermoplastic polymers may also be used to form the elastic film (if the liquid-permeable substrate is to comprise an elastic film) so long as they do not adversely affect the elastic properties of the film (or as discussed elsewhere, e.g., strands, or composites). For example, the elastic film may contain other polyolefins, elastomeric polyesters, polyurethanes, polyamides, block copolymers, and so forth. For example, polyethylene may be employed in some embodiments of the present invention. The density of the polyethylene may vary depending on the type of polymer employed, but generally ranges from 0.85 to 0.96 grams per cubic centimeter (“g/cm³”). Polyethylene “plastomers”, for instance, may have a density in the range of from 0.85 to 0.91 g/cm³. Likewise, “linear low density polyethylene” (“LLDPE”) may have a density in the range of from 0.91 to 0.940 g/cm³; “low density polyethylene” (“LDPE”) may have a density in the range of from 0.910 to 0.940 g/cm³; and “high density polyethylene” (“HDPE”) may have density in the range of from 0.940 to 0.960 g/cm³.

Besides polymers, an elastic film, if employed in the present invention, may also contain other additives as is known in the art, such as melt stabilizers, processing stabilizers, heat stabilizers, light stabilizers, antioxidants, heat aging stabilizers, whitening agents, antiblocking agents, bonding agents, tackifiers, viscosity modifiers, etc. Suitable viscosity modifiers may include, for instance, polyethylene wax (e.g., EPOLENE™ C-10 from Eastman Chemical). Phosphite stabilizers (e.g., IRGAFOS available from Ciba Specialty Chemicals of Terrytown, N.Y. and DOVERPHOS available from Dover Chemical Corp. of Dover, Ohio) are exemplary melt stabilizers. In addition, hindered amine stabilizers (e.g., CHIMASSORB available from Ciba Specialty Chemicals) are exemplary heat and light stabilizers. Further, hindered phenols are commonly used as an antioxidant in the production of films. Some suitable hindered phenols include those available from Ciba Specialty Chemicals of under the trade name “Irganox®”, such as Irganox®1076, 1010, or E 201. Moreover, bonding agents may also be added to the film to facilitate bonding of the film to additional materials (e.g., nonwoven web). When employed, such additives (e.g., tackifier, antioxidant, stabilizer, etc.) may each be present in an amount from about 0.001 wt. % to about 25 wt. %, in some embodiments, from about 0.005 wt. % to about 20 wt. %, and in some embodiments, from 0.01 wt. % to about 15 wt. % of the film. These (and other) components can be mixed together, heated and then extruded into a mono-layer or multi-layer film using any one of a variety of film-producing processes known to those of ordinary skill in the film processing art. Such film-making processes include, for example, cast embossed, chill and flat cast, and blown film processes.

As discussed elsewhere, the liquid-permeable substrate comprising a film (whether the film possesses elastic properties or not) may be apertured or perforated. Such perforations or apertures may be performed by methods known in the art such as for example slit aperturing or pin aperturing with heated or ambient temperature pins.

In some embodiments, the porous or apertured film is attached to an outer fibrous layer. The outer fibrous layer can give the appliance a cloth-like feel and appearance. If the film, with or without an outer fibrous layer, is attached to an inner fibrous layer, undulations in the inner fibrous layer (which contacts skin or tissue of the user) helps contain the formulation or composition that is applied to, or associated with, the inner fibrous layer. As discussed elsewhere, undulations or texture may be effected, in whole or in part, by attaching the outer- and/or inner-fibrous layer to the liquid-permeable film at discrete points or locations while the liquid-permeable film is in a stretched condition. When the resulting laminate is allowed to contract, the undulations or texture are associated with the outer- and/or inner-fibrous layer. Furthermore, to the extent the liquid-permeable film employs elastomeric materials, the resulting appliance is able to stretch and more readily conform to the skin surface. And if any optional inner- and/or outer fibrous layer is bonded at discrete locations to the elastomeric liquid-permeable layer while in a stretched condition, and then allowed to retract, thereby partially gathering the fibrous layer(s), then the appliance is especially well suited to being adapted to stretch and conform to complex skin surfaces.

In other embodiments of the invention, the liquid-permeable substrate is a fibrous substrate, such as a nonwoven material. Other versions of the present invention encompass an appliance comprising an elastomeric composite having individual elastic filaments, strands, or fiber (e.g., substantially parallel, spaced-apart strands attached to meltblown fiber). The elastomeric composite itself may be attached to one or more fibrous layers (e.g., the elastomeric composite may be sandwiched between an inner and outer fibrous layer).

### Representative Substrates for Constructing an Appliance of the Present Invention

A liquid-permeable substrate employed in an appliance of the present invention will generally comprise an apertured or porous film (with or without any optional fibrous layers); a fibrous layer; an elastomeric composite having individual elastic strands, filaments, or the like; or other liquid-permeable material.

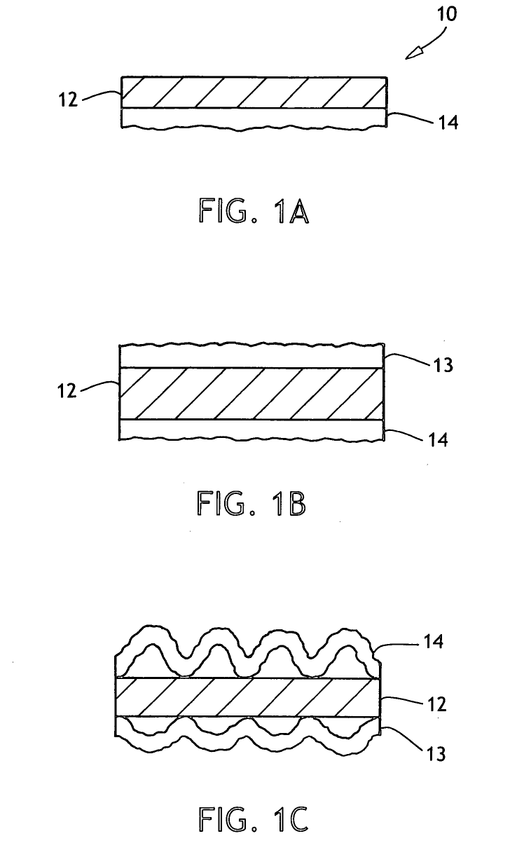

An example of such a substrate 10 is depicted in FIG. 1, which representatively illustrates a porous film 12 attached to a fibrous layer 14. A suitable class of film materials includes a thermoplastic elastomeric polyolefin polymer. As note above, these (and other) components can be mixed together, heated, and then extruded into a mono-layer or multi-layer film using any one of a variety of film-producing processes known to those of ordinary skill in the film processing art. Such film-making processes include, for example, cast embossed, chill and flat cast, and blown film processes. Typically the film layer 12 will be attached to any optional fibrous layer 14 using an adhesive, thermal bonding, ultrasonic bonding, or the like. As noted elsewhere, the film can be perforated or apertured to make the film liquid-permeable.

Other additives and ingredients may be added to the film 12 provided they do not significantly interfere with the ability of the film to function in accordance with the teachings of the present invention. Such additives and ingredients can include, for example, antioxidants, stabilizers, and pigments.

In addition to the polyolefin polymer in this representative example, a film 12 can also include a filler. As used herein, a “filler” is meant to include particulates and other forms of materials that can be added to the film polymer extrusion blend and that will not chemically interfere with the extruded film but which are able to be uniformly dispersed throughout the film. Generally, the fillers will be in particulate form and may have a spherical or non-spherical shape with average particle sizes in the range of about 0.1 to about 7 microns. Both organic and inorganic fillers are contemplated to be within the scope of the present invention provided that they do not interfere with the film formation process, or the ability of the film layer to function in accordance with the teachings of the present invention. Examples of suitable fillers include calcium carbonate (CaCO₃), various kinds of clay, silica (SiO₂), alumina, barium carbonate, sodium carbonate, magnesium carbonate, talc, barium sulfate, magnesium sulfate, aluminum sulfate, titanium dioxide (TiO₂), zeolites, cellulose-type powders, kaolin, mica, carbon, calcium oxide, magnesium oxide, aluminum hydroxide, pulp powder, wood powder, cellulose derivatives, chitin and chitin derivatives. A suitable coating, such as, for example, stearic acid, may also be applied to the filler particles.

As mentioned herein, the film 12 may be formed using any one of the conventional processes known to those familiar with film formation, e.g., a polyolefin or other polymer and any optional ingredients (e.g., filler) are mixed in and then heated and extruded into a film.

If a nonwoven material is used to make the fibrous layer, then commercially available thermoplastic polymeric materials can be advantageously employed in making the fibers or filaments from which the fibrous layer 14 is formed. As used herein, the term “polymer” shall include, but is not limited to, homopolymer, copolymers, such as, for example, block, graft, random and alternating copolymers, terpolymers, etc., and blends and modifications thereof. Moreover, unless otherwise specifically limited, the term “polymer” shall include all possible geometric configurations of the material, including, without limitation, isotactic, syndiotactic, random and atactic symmetries. As used herein, the terms “thermoplastic polymer” or “thermoplastic polymeric material” refer to a long-chain polymer that softens when exposed to heat and returns to the solid state when cooled to ambient temperature. Exemplary thermoplastic materials include, without limitation, polyvinyl chlorides, polyesters, polyamides, polyfluorocarbons, polyolefins, polyurethanes, polystyrenes, polyvinyl alcohols, caprolactams, and copolymers of the foregoing.

Nonwoven webs that can be employed as fibrous layer 14 of the present invention can be formed by a variety of known forming processes, including spunbonding, airlaying, meltblowing, or bonded carded web formation processes. Spunbond nonwoven webs are made from melt-spun filaments. As used herein, the term “meltspun filaments” refers to small diameter fibers and/or filaments which are formed by extruding a molten thermoplastic material as filaments from a plurality of fine, usually circular, capillaries of a spinneret with the diameter of the extruded filaments then being rapidly reduced, for example, by non-eductive or eductive fluid drawing or other well known spunbonding mechanisms. Lastly, the melt-spun filaments are deposited in a substantially random manner onto a moving carrier belt or the like to form a web of substantially continuous and randomly arranged, melt-spun filaments. Spunbond filaments generally are not tacky when they are deposited onto the collecting surface. The production of spunbond nonwoven webs is described in U.S. Pat. No. 4,340,563 to Appel et al., U.S. Pat. No. 3,692,618 to Dorschner et al., U.S. Pat. No. 3,802,817 to Matsuki et al., U.S. Pat. Nos. 3,338,992 and 3,341,394 to Kinney, U.S. Pat. No. 3,502,538 to Peterson, and U.S. Pat. No. 3,542,615 to Dobo et al., all of which are incorporated herein by reference. The melt-spun filaments formed by the spunbond process are generally continuous and have average diameters larger than 7 microns based upon at least 5 measurements, and more particularly, between about 10 and 100 microns. Another frequently used expression of fiber or filament diameter is denier, which is defined as grams per 9000 meters of a fiber or filament.

Spunbond webs generally are stabilized or consolidated (pre-bonded) in some manner immediately as they are produced in order to give the web sufficient integrity and strength to withstand the rigors of further processing into a finished product. This pre-bonding step may be accomplished through the use of an adhesive applied to the filaments as a liquid or powder which may be heat activated, or more commonly, by compaction rolls. As used herein, the term “compaction rolls” means a set of rollers above and below the nonwoven web used to compact the web as a way of treating a just produced, melt-spun filament, particularly spunbond, web, in order to give the web sufficient integrity for further processing, but not the relatively strong bonding of later applied, secondary bonding processes, such as through-air bonding, thermal bonding, ultrasonic bonding and the like. Compaction rolls slightly squeeze the web in order to increase its self-adherence and thereby its integrity.

An exemplary secondary bonding process utilizes a patterned roller arrangement for thermally bonding the spunbond web. The roller arrangement typically includes a patterned bonding roll and a smooth anvil roll which together define a thermal patterning bonding nip. Alternatively, the anvil roll may also bear a bonding pattern on its outer surface. The pattern roll is heated to a suitable bonding temperature by conventional heating means and is rotated by conventional drive means, so that when the spunbond web passes through the nip, a series of thermal pattern bonds is formed. Nip pressure within the nip should be sufficient to achieve the desired degree of bonding of the web, given the line speed, bonding temperature and materials forming the web. Percent bond areas within the range of from about 10 percent to about 20 percent are typical for such spunbond webs.

As noted above, the film 12 will typically be attached to any optional fibrous layer 14 by thermally bonding the layers together at discrete points (see, e.g., above discussion as well as U.S. Pat. No. 6,037,281, entitled “Cloth-Like, Liquid-Impervious, Breathable Composite Barrier Fabric,” to Mathis, et al.); with or without adhesive; or by ultrasonic methods; or other such approaches. As noted above, the fibrous layer may be bonded or attached to the film at discrete locations while the film is in a stretched condition, thereby producing or increasing undulations when the resulting laminate is in a relaxed condition. Other known means for bonding and laminating the film 12 to a fibrous layer may be used, provided the resulting substrate 10 has the required properties described herein.

If a porous or apertured film is attached to a fibrous layer on one side of the film, then the finished appliance may be adapted for use with the fibrous layer oriented toward the skin or tissue of a user (i.e., with the substantially dry formulation associated with the fibrous layer and available for transfer to skin or tissue in contact with the fibrous layer); or with the fibrous layer oriented away from the skin or tissue of a user (i.e., with the substantially dry formulation associated with the film and available for transfer to skin or tissue in contact with the film).

Also, as noted above, and as depicted in FIG. 1B, a porous or apertured film 12 may be attached to a fibrous layer on both sides or faces of the film. In this case the appliance will employ a fibrous layer next to the skin or tissue of a user of the appliance, and facing outward away from the skin. The outer fibrous layer 13 can give the appliance a soft, cottony appearance. And the inner fibrous layer 14 can help retain the formulation (i.e., help reduce leakage because the fibrous layer possesses a pore structure and/or rugosity that helps keep the formulation, which, as noted elsewhere, is typically a solid or semi-solid, associated with the appliance).

The layer 12 may also be a composite. For example, layer 12 can be a composite comprising elastic strands or filaments, optionally attached to a meltblown layer. In one version of the present invention, the composite has spaced-apart, substantially parallel strands attached to meltblown fiber.

FIG. 1C depicts another representative version of the invention. A central layer 12, such as an apertured or porous film, or a composite—such as a composite having elastic strands and meltblown fiber, is sandwiched between an outer fibrous layer 13 and an inner fibrous layer 14. While the description associated with the preceding Figure noted that an inner and/or outer fibrous layer could be gathered (without the Figure actually depicting what one version of a gathered layer looks like), FIG. 1C depicts a substrate having gathered layers. Here the apertured film or composite 12 is bonded at discrete locations to an outer fibrous layer 13, and to an inner fibrous layer 14. One way to make the substrate depicted in FIG. 1C is to stretch the apertured film or composite 12 prior to its being directed to a nip between two rolls employed to bond the film or composite 12 to the other layers. So, for example, if film or composite 12 is an elastomeric apertured film, then the film is stretched prior to its being directed to the aforementioned nip. To make the embodiment depicted in FIG. 1C, two additional webs are also directed to the same nip: the outer fibrous layer 13 (e.g., a necked polypropylene spunbond material) on one side of the film and an inner fibrous layer 14 (e.g., a necked polypropylene spunbond material). By bonding the outer fibrous layer 13 and inner fibrous layer 14 intermittently along the length of the film or composite 12, and then allowing the resulting combination to contract, a substrate like that depicted in FIG. 1C is formed. Both the outer fibrous layer and inner fibrous layer are gathered: i.e., at certain locations along the length of the film or composite 12 the outer fibrous layer and the inner fibrous layer are adjacent to the film or composite, and are bonded to the film or composite. Between these points or regions of adjacency/attachment, the outer fibrous layer and the inner fibrous layer are not attached to the film or composite, and these unattached portions may appear like “hills” or undulations between the points of attachment.

It should be noted that various patterns may be used when thermally, adhesively, or otherwise bonding two or more layers together. While FIG. 1C provides what is, in effect, a side-view of a cross-section of the substrate, a top-down view would reveal a pattern of the discrete bonding locations by which the two or more layers are attached to one another. For example, a top down view could show discrete bonding areas that resemble the letter “Y”, with unbonded regions around each of these discrete Y-shaped bonded areas.

An appliance of the present invention need not employ a film. An appliance may comprise one or more fibrous layers and a substantially dry formulation associated with said fibrous layer(s). And the appliance may be constructed of other fibrous layers, apertured or porous films, or other materials, so long as the resulting appliance is liquid permeable.

The preceding paragraphs describe some examples of a substrate that may be used in an appliance of the present invention. For additional examples, see, e.g., U.S. Pat. No. 6,037,281, entitled “Cloth-Like, Liquid-Impervious, Breathable Composite Barrier Fabric,” to Mathis, et al.; U.S. Pat. No. 4,663,220 issued May 5, 1987 to Wisneski et al.; U.S. Pat. No. 5,226,992 issued Jul. 13, 1993 to Morman; European Patent Application No. EP 0 217 032 published on Apr. 8, 1987 in the name of Taylor et al.; and PCT Patent Application WO 01/88245 in the name of Welch et al.; all of which are incorporated herein by reference in a manner consistent herewith.

### Representative Appliance Configurations

One or more substrates, such as those described above, may be configured into the form of a glove, mitten, sock, sleeve, patch, or other article designed to be fitted to a part of the body. Generally the appliance will be made by cutting a substrate into appropriate pieces such that the pieces, when attached to one another, form an appliance having an interior volume into which a portion of a body may be inserted. The interior volume is bounded, at least in part, by an interior, body-facing surface. But, as noted above, the appliance may be configured in the form of a patch. The patch would have a body-facing surface. Typically a formulation will be associated with the appliance during manufacture (either during or after: manufacture of one or more layers of the liquid-permeable substrate; converting of the substrate into an appliance such as a sock or glove; etc.—this may be accomplished in one manufacturing line or in separate lines or steps, some of which may be performed at different geographic locations) so that the appliance is ready to use.

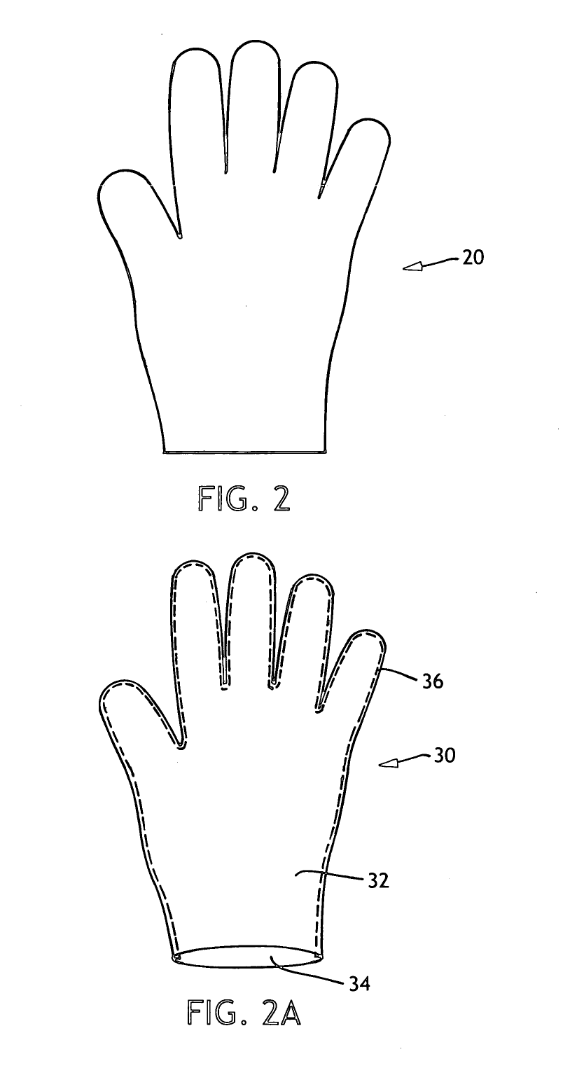

FIG. 2 representatively depicts a substrate 20 cut so that the piece (or substrate) defines a perimeter in the shape of a human hand. FIG. 2A representatively depicts an appliance 30 comprising a first piece (or substrate) 32 attached to a second piece (or substrate) 34 at a location proximate to the perimeters of these two substrates. In this representative illustration, the two substrates are attached to one another mechanically by sewing the pieces together at a location proximate to the perimeters of the two substrates. The resulting appliance was then inverted so that the seam 36 formed by sewing the substrates together is on the interior of the appliance. Of course the finished appliance need not be inverted; the seam can remain on the exterior of the appliance. Note, too, that the individual pieces need not be joined in a way that produces a seam. The edges of the individual pieces may be butted together, and then, for example, joined and/or welded together using a solvent. Alternatively, the individual pieces may be butted together, and another material, such as an adhesive or an adhesive tape, used to join the pieces together. Or the pieces may be thermally bonded or ultrasonically bonded. Furthermore, any glove-like appliance may be formed such that the appliance resembles a bicycle glove, or some portion thereof (i.e., one or more end portions of the individual thumb-like and/or finger-like projections of the glove-like appliance are absent, so that a person may more easily manipulate objects while wearing the appliance because some portion of one or more fingers and/or the thumb is exposed [and at the same time treat skin, for example, at joints, the back of the hand, the palm, or some combination thereof]). Alternatively, a sock may be formed such that a portion proximate to the heel, the toe(s), or some other portion of a user's foot is exposed.

Individual pieces (or substrates) may be cut into a variety of shapes and sizes. Rather than the glove depicted in FIGS. 2 and 2A, the pieces may be cut so that the resulting appliance is in the shape of a tube, sleeve, mitten, sock, or the like. Any shape is possible, so long as the resulting appliance defines an interior volume (for those versions of the invention in which the appliance defines an interior volume) into which a user may insert a portion of his or her body (e.g., a finger, toe, hand, foot, wrist, forearm, etc.) such that a composition applied to, or associated with, the interior surface of the appliance may be brought into contact with skin or tissue proximate to the interior surface of the appliance. As noted elsewhere, however, in some versions of the invention the appliance is a patch that is applied or affixed to skin (e.g., a patch comprising a body adhesive proximate to the perimeter of the patch, thereby allowing the patch to be releasably affixed to the skin).

The individual substrates or pieces need not be sewn together. The individual pieces or substrates may also be joined ultrasonically, thermally, adhesively, cohesively, using tape, by fusing the materials together (e.g., by using an appropriate solvent), by welding the materials together, or by other approaches. So long as the individual pieces or substrates remain attached or connected during normal use of the appliance, and attachment or connection is such that any composition or formulation on the interior surface of the appliance is contained within the appliance (i.e., there is minimal or no leakage of the formulation or composition), any connection or attachment may be used.

Alternatively, a substrate could be prepared in the form of a rectangle, oval, or other shape (e.g., as for a patch). An adhesive capable of adhering to skin could then be applied to all or part of the perimeter of the shape such that the appliance could be releasably adhered to skin. Any composition to be brought into contact with skin could then be coated or deposited on the surface of the appliance that will contact skin or tissue.

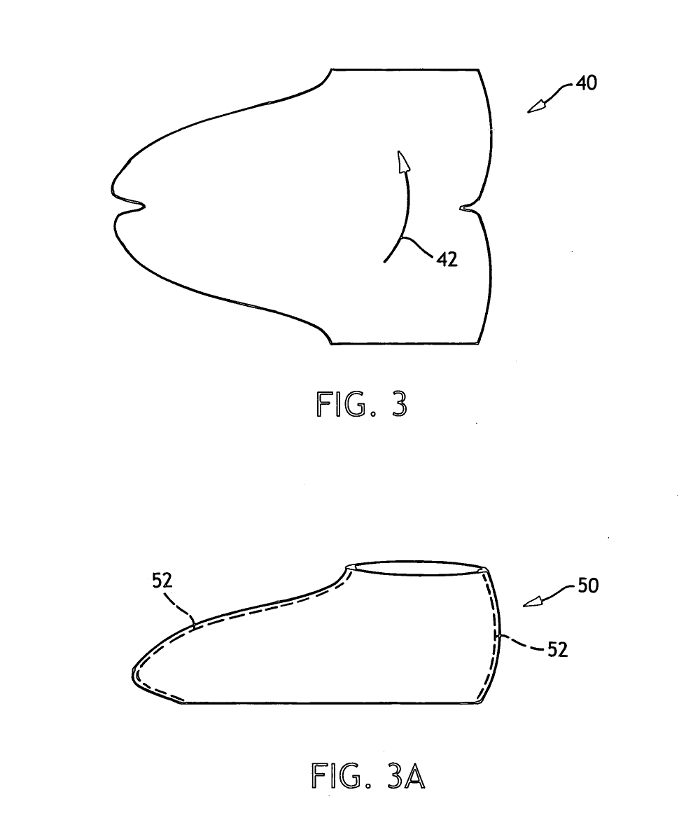

Note, too, that an appliance defining some interior volume may be formed from a single piece of substrate. In one exemplary embodiment, FIG. 3 representatively illustrates a substrate 40 that has been cut in a way that a foot-shaped appliance may be formed by folding the substrate back on itself (as shown by arrow 42; the bottom half of the shape is folded upward, and on top of, the top half of the shape). FIG. 3A representatively illustrates such a foot-shape appliance 50 and the resulting seams 52 formed when the substrate 40 (from FIG. 3) is folded back, and attached to, itself. In this representative embodiment, the foot-shape appliance was inverted after the substrate was attached to itself so that the seams were on the inside of the appliance. As with two (or more) pieces that may be joined together to form an appliance of the present invention, a single piece may be joined to itself using any of the approaches discussed above (e.g., to form the foot appliance, a sleeve, etc.).

### Representative Formulations or Compositions for Use with an Appliance of the Present Invention

Formulations or compositions that may be used with an appliance of the present invention include emulsifiers, surfactants, viscosity modifiers, natural moisturizing factors, antimicrobial actives, pH modifiers, enzyme inhibitors/inactivators, suspending agents, pigments, dyes, colorants, buffers, perfumes, antibacterial actives, antifungal actives, pharmaceutical actives, film formers, deodorants, opacifiers, astringents, solvents, organic acids, preservatives, drugs, vitamins, aloe vera, and the like.

Formulations or compositions employed in appliances of the present invention may be hydrophilic in nature (but need not be so—the composition may in fact be hydrophobic); that is, the compositions are attracted to, and retain, water. One measurement of a composition's hydrophilicity is the composition's ability to dissolve or disperse in water. Typically, such dissolution/dispersion evaluations are done with heated water to improve the rate of dissolution/dispersion. The more soluble a composition is in water (typically compositions may be tested with water at a temperature of about 80° Celsius [or, when preceded by the term degrees or its corresponding symbol, “C”] as the formulations may be solid at room temperature) the more hydrophilic the composition.

Hydrophilic formulations employed with appliances of the present invention are often at least about 40%, typically at least about 45%, suitably at least about 60%, particularly at least about 70%, and still more suitably at least about 80% soluble/dispersible in deionized water at a temperature of about 80° C. A typical hydrophilic composition of the present invention is about 80% soluble/dispersible in deionized water at a temperature of about 80° C.

Formulations of the present invention generally have a melting point of from about 30° C. to about 80° C., suitably from about 40° C. to about 70° C., and particularly from about 55° C. to about 75° C. With melting points in these ranges, the formulations employed on appliances of the present invention have a reduced tendency to flow easily and migrate through the liquid-impermeable substrate (which, as discussed elsewhere, may comprise an apertured or porous film, a fibrous layer, strands or filaments, a plurality of one or more of the preceding materials, laminates of one or more of the preceding materials, some combination of the aforementioned single- or multiple-layer materials, and the like). This is important for at least two reasons. First, it is preferred that the majority of the composition remain associated with the surface of the appliance to allow direct interaction with the skin or mucous membrane it contacts. As such, a sufficiently high freezing temperature is desired such that the composition freezes, or solidifies, onto the substrate to which it is applied in a short amount of time to reduce the potential for run-off or migration. Therefore, the composition or formulation typically freezes onto the substrate in no more than about 3 seconds, suitably no more than about 0.25 seconds. With freezing rates in these ranges, compositions employed on appliances of the present invention solidify quickly onto the substrate and the potential for migration is minimized (i.e., the formulation or composition is substantially immobile).

Secondly, the melting point needs to be sufficiently high to provide sufficient stability for the composition. Stated another way, the melting points are desirably higher than the temperatures that the appliance to which the composition has been applied is exposed to during storage and transport, which may be as high as about 55° C.

Additionally, the compositions employed with appliances of the present invention often have a penetration hardness (needle penetration in millimeters according to ASTM D 1321, “Needle Penetration of Petroleum Waxes”) of from about 1 millimeter to about 200 millimeters, typically from about 1 millimeter to about 120 millimeters, suitably from about 1 millimeter to about 20 millimeters, and particularly from about 3 to about 17 millimeters. Penetration hardness of the compositions may be important for two reasons. First, the softer the formulation (i.e., the higher the penetration hardness number) the more mobile the formulation will be, making the formulation more likely to migrate. As such, it is typically desirable to have a penetration hardness of not more than 50 millimeters, suitably not more than about 20 millimeters to reduce this likelihood.

Second, very soft formulations tend to be more greasy/oily to the touch, which is typically not desirable. By requiring compositions employed with appliances of the present invention to have a penetration hardness as described above, these compositions are less likely to migrate yet maintain a silky, creamy feeling on the appliance.

Compositions employed with appliances of the present invention are preferably substantially non-irritating to the skin or mucous membrane to which they contact during use by the wearer; that is, it is preferred that the moisturizing and lubricating compositions not induce redness and/or swelling of the skin tissues when contacted with the skin of the wearer. Further, it is preferred that the compositions not interfere with or hinder the natural ability of the skin and mucous membranes to repair themselves from injury due to, for example, wiping or other abrasive activity. As discussed below, some of the compositions of the present invention may contain a fat or oil and/or a sterol or sterol derivative to facilitate repair of the skin from such damage.

Compositions employed with appliances of the present invention typically comprise an emollient, a humectant, an immobilizing agent, a compatibilizing agent, and optionally a dispersing agent. Other optional components may also be included in compositions described herein.

Compositions used with the present invention typically comprise from about 1% (by weight) to about 40% (by weight) of an emollient. As used herein, the term “by weight” refers to the total weight of the composition. Thus, if a composition is 25% (by weight) emollients and has a total weight of 100 grams, the compositions comprises 25 grams of emollient. As used herein, an emollient refers to a compound that smoothes, softens, soothes, supples, coats, lubricates, moisturizes, protects and/or cleanses the skin upon contact.

Emollients suitable for use in compositions used with the present invention include, but are not limited to, petroleum based emollients, fatty acids, fatty acids esters, vegetable oils, hydrogenated vegetable oils, alkyl ethoxylates, fatty alcohols and silicones such as dimethicone, dimethiconol, PEG dimethicone, alkyl silicones, phenyl silicones, and silicone phospholipids, and combinations thereof. Additional examples of emollients used with the present invention include silicone based emollients such as Dow Corning 200 Fluids and Dow Corning 1503 Fluids.

Suitable petroleum based emollients include those hydrocarbons, or mixtures of hydrocarbons, having chain lengths of from 16 to 32 carbon atoms. Petroleum based hydrocarbons having these chain lengths include mineral oil (also known as “liquid petrolatum”) and petrolatum (also known as “mineral wax,” “petroleum jelly” and “mineral jelly”). Mineral oil usually refers to less viscous mixtures of hydrocarbons having from 16 to 20 carbon atoms. Petrolatum usually refers to more viscous mixtures of hydrocarbons having from 16 to 32 carbon atoms.

Suitable fatty acid ester emollients include those derived from C₁₂-C₂₈ fatty acids, preferably C₁₆-C₂₂ saturated fatty acids, and short chain, such as C₁-C₈, preferably C₁-C₃, monohydric alcohols. Examples include methyl palmitate, methyl stearate, isopropyl laurate, isopropyl myristate, butyl myristate, butyl stearate, octyl palmitate, isopropyl isostearate, isopropyl palmitate, ethylhexyl palmitate, and mixtures thereof. Suitable fatty acid ester emollients can also be derived from monoesters and diesters of both short chain, such as C₁-C₁₀, and longer chain fatty alcohols, such as C₁₂-C₂₈, preferably C₁₂-C₁₆, and shorter chain organic acids such as lactic acid, lauryl lactate and cetyl lactate. Additional examples include diisopropyl sebacate, dimethyl sebacate, dioctyl sebacate, dibutyl sebacate, diisopropyl adipate, and dicapryl adipate. In addition, mixtures of petroleum based emollients and fatty acid ester emollients can provide emollient systems that have a superior feel compared to the pure components individually.

Suitable alkyl ethoxylate type emollients include C₁₂-C₂₂ fatty alcohol ethoxylates having an average degree of ethoxylation of from about 2 to about 30. Particularly, the fatty alcohol ethoxylate emollient is selected from the group of lauryl, cetyl, and stearyl ethoxylates, and mixtures thereof, having an average degree of ethoxylation ranging from about 2 to about 23. Representative examples of such alkyl ethoxylates include laureth-3 (a lauryl ethoxylate having an average degree of ethoxylation of 3), laureth-23 (a lauryl ethoxylate having an average degree of ethoxylation of 23), ceteth-10 (a cetyl alcohol ethoxylate having an average degree of ethoxylation of 10) steareth-10 (a stearyl alcohol ethoxylate having an average degree of ethoxylation of 10), and ceteareth-10 (a mixture of cetyl and stearyl ethoxylates having an average degree of ethoxylation of 10). Additionally, alkyl ethoxylates with an HLB of from about 7 to about 14 are also useful as emulsifiers or compatibilizers/solubilizers of other emollients in the composition.

Suitable fatty acid-type emollients include acids having a carbon chain length of C₁₄-C₃₀ including myristic acid, palmitic acid, stearic acid, behenic acid, and mixtures thereof. Additionally, C₉-C₁₅ acids including caprylic acid, lauric acid, and the like are suitable fatty acid-type emollients.

Suitable fatty alcohol-type emollients include alcohols having a carbon chain length of C₁₄-C₃₀, including cetyl alcohol, stearyl alcohol, arachidyl alcohol, and behenyl alcohol and mixtures thereof. Additionally, C₉-C₁₅ alcohols including caprylic alcohol, cetearyl alcohol, coconut alcohol, decyl alcohol, hydrogenated tallow alcohol, lanolin alcohol, lauryl alcohol, myristyl alcohol, oleyl alcohol, palm alcohol, palm kernel alcohol, tallow alcohol, tridcyl alcohol and the like are suitable fatty alcohol-type emollients.

Another suitable type of emollient is a silicone, such as a polysilioxane compound. Generally, suitable polysiloxane materials for use include those having monomeric siloxane units having the following structure: wherein x is a whole number from 1 to about 1,000,000 and R₁ and R₂ for each independent siloxane monomeric unit can each independently be hydrogen or any alkyl, aryl, alkenyl, alkaryl, arakyl, cycloalkyl, halogenated hydrocarbon, or other radical. Any of such radicals can be substituted or unsubstituted. R₁ and R₂ radicals of any particular monomeric unit may differ from the corresponding functionalities of the next adjoining monomeric unit. Additionally, the polysiloxane can be either a straight chain, a branched chain or have a cyclic structure. The radicals R₁ and R₂ can additionally independently be other silaceous functionalities such as, but not limited to siloxanes, polysiloxanes, silanes, and polysilanes. The radicals R₁ and R₂ may contain a variety of organic functionalities including, for example, alcohol, carboxylic acid, phenyl, and amine functionalities.

Exemplary radicals are methyl, ethyl, propyl, butyl, pentyl, hexyl, octyl, decyl, octadecyl, and the like. Exemplary alkenyl radicals are vinyl, allyl, and the like. Exemplary aryl radicals are phenyl, diphenyl, naphthyl, and the like. Exemplary alkaryl radicals are toyl, xylyl, ethylphenyl, and the like. Exemplary aralkyl radicals are benzyl, alphaphenylethyl, beta-phenylethyl, alpha-phenylbutyl, and the like. Exemplary cycloalkyl radicals are cyclobutyl, cyclopentyl, cyclohexyl, and the like. Exemplary halogenated hydrocarbon radicals are chloromethyl, bromoethyl, tetrafluorethyl, fluorethyl, trifluorethyl, trifluorotoyl, hexafluoroxylyl, and the like.

The viscosity of the useful polysiloxanes may vary widely. So long as the polysiloxane is flowable or can be made to be flowable for application, the polysiloxane viscosity is acceptable. This includes, but is not limited to, viscosity as low as 5 centistokes (at 37° C. as measured by a glass viscometer) to about 20,000,000 centistokes (at 37° C. as measured by a glass viscometer). A typical range is from about 5 centistokes to about 5,000 centistokes. In one embodiment, a mixture of a low viscosity dimethicone (about 5 centistokes to about 350 centistokes) and a high viscosity dimethicone or dimethiconol or silicone gum (1,000,000 centistokes to about 20,000,000 centistokes) is used as the mixture produces a pleasant feeling material and provides additional lubricity. Suitable ranges of the low viscosity polysiloxane to high viscosity polysiloxane is from about 1:1 to about 10:1.

Polysiloxane compounds suitable for use as emollients in the present invention are disclosed in U.S. Pat. No. 5,059,282 (Ampulski, et al.). Other polysiloxane compounds for use as emollients in compositions used with the present invention include phenyl-functional polymethylsiloxane compounds (e.g., Dow Corning 556 Cosmetic-Grade Fluid) and cetyl or stearyl functionalized dimethicones such as Dow 2502, General Electric SF1632 and Dow 2503 polysiloxane fluids. In addition to such substitution with phenyl-functional or alkyl groups, effective substitution may be made with amino, carboxyl, hydroxyl, ether, polyether, aldehyde, ketone, amide, ester, and thiol groups. Phenyl, amino, alkyl, carboxyl, and hydroxyl groups are preferred, with phenyl functional groups being most preferred.

The humectant component of compositions of the present invention are generally present in an amount of from about 1% (by weight) to about 20% (by weight), typically from about 5% (by weight) to about 15% (by weight). Humectants are typically cosmetic ingredients used to increase the water content of the top layers of the skin or mucous membrane, by helping control the moisture exchange between the product, the skin, and the atmosphere. Humectants may include primarily hydroscopic materials. Suitable humectants for inclusion in the compositions of the present invention include urocanic acid, N-Acetyl ethanolamine, aloe vera gel, arginine PCA, chitosan PCA, copper PCA, Corn glycerides, dimethyl imidazolidinone, fructose, glucamine, glucose, glucose glutamate, glucuronic acid, glutamic acid, glycereth-7, glycereth-12, glycereth-20, glycereth-26, glycerin, honey, hydrogenated honey, hydrogenated starch hydrolysates, hydrolyzed corn starch, lactamide MEA, lactic acid, lactose lysine PCA, mannitol, methyl gluceth-10, methyl gluceth-20, PCA, PEG-2 lactamide, PEG-10 propylene glycol, polyamino acids, polysaccharides, polyamino sugar condensate, potassium PCA, propylene glycol, propylene glycol citrate, saccharide hydrolysate, saccharide isomerate, sodium aspartate, sodium lactate, sodium PCA, sorbitol, TEA-lactate, TEA-PCA, Urea, Xylitol, and the like and mixtures thereof. Suitable humectants include polyols, glycerine, ethoxylated glycerine, polyethylene glycols, hydrogenated starch hydrolsates, propylene glycol, silicone glycol and pyrrolidone carboxylic acid.

The immobilizing agent component of compositions used with the present invention are generally present in an amount of from about 30% (by weight) to about 90% (by weight), suitably from about 40% (by weight) to about 70% (by weight). The immobilizing agent will reduce the tendency of the emollient and humectant to migrate or flow by keeping the emollient and humectant primarily localized. In addition to immobilizing the emollient and humectant, the immobilizing agent may provide a slight tackiness to the composition, which may improve transfer of the composition to skin or membrane of the wearer.

Suitable immobilizing agents for use with compositions used with the present invention include metal soaps including aluminum, calcium, magnesium, and zinc stearates, C₁₄-C₂₂ fatty alcohols, C₁₂-C₂₂ fatty acids, solid fatty acid esters, C₁₂-C₂₂ fatty alcohol ethoxylates having an average degree of ethoxylation ranging from about 2 to about 30, and high molecular weight (greater than about 720) polyethylene glycols (a polymer of ethylene oxides) that are solids at room temperature having the following empirical formula:

H(OCH₂CH₂)ₓOH

wherein x is the degree of ethoxylation and is an average value of at least about 20 moles or greater. Suitably, x is an average value of from about 20 to about 1,000, and even more suitably from about 100 to about 500. Other useful high molecular weight polyethylene glycols are polyethylene glycols having the technical names of PEG 1000 (where x=20), PEG 3350 (where x=75), PEG 6000 (where x=125), PEG 8000 (where x=150), and PEG 10,000 (where x=220).

Suitable fatty alcohol immobilizing agents include C₁₆-C₁₈ fatty alcohols and fatty acids such as myristyl, cetyl, cetearyl, stearyl, behenyl alcohols and acids, and mixtures thereof. These immobilizing agents increase the rate of crystallization of the emollient causing the emollient to crystallize rapidly.

Along with the emollient, humectant, and immobilizing agent, the compositions described herein comprise a compatibilizing agent. Compatibility of the overall composition is useful for processability and stability. Incompatible compositions require a more rigorous process to ensure that mixing is complete so as to prevent the separation of the different components in the composition. More mixing requires higher energy consumption, which leads to an increase in the cost of manufacturing the products. Further, it may be very difficult for an incompatible composition to maintain acceptable stability during the life of the product, starting with shipping, transportation, and storage prior to ultimate use by the consumer. Many incompatible ingredients may tend to slowly separate from the surface of the appliance to which they are applied resulting in a loss of the properties of the overall composition and a potential loss in the intended benefits.

Some components described herein as components of compositions used with the present invention may be incompatible with the humectant glycerin. Specifically, it has been discovered that several immobilizing agents, including high molecular weight polyethylene glycols, are actually incompatible with glycerin. As such, in order to ensure a high degree of compatibility and a substantially homogeneous composition, the compositions described herein include a compatibilizing agent. For example, the compatibilizing agent is capable of compatibilizing glycerin and high molecular weight polyethylene glycols. The compatibilizing agent may be selected from propylene glycol, butylene glycol, 1,3 butylene glycol, low molecular weight polyethylene glycols (molecular weights of less than about 720 and liquid at room temperature such as, for example, PEG 600), methoxyisopropanol, dipropylene glycol propyl ether, dipropylene glycol butyl ether, dipropylene glycol, methyl propanediol, and soluble/dispersible polypropylene glycols. The compatibilizing agent is present in the compositions employed with present invention in an amount of from about 1% (by weight) to about 40% (by weight).

As mentioned above, the compositions described herein may optionally comprise a dispersing agent. Because some silicones, which may be introduced into the compositions as emollients as discussed above, may be incompatible with glycerin and some glycols, a dispersing agent may be added to improve the compatibility of silicones when they are introduced into the composition. Useful dispersing agents include polyether ethoxylated/propoxylated modified polydimethylsiloxanes which are fully or partially compatible with polydimethylsiloxanes, silicone polyethers having at least 30% siloxane, between 10% and 40% ethoxylation and between 0% and 40% propoxylation. For example, Dow Corning 5329 may be introduced as a dispersing agent to obtain the desired benefits. The weight ratio of silicone dispersing agent to silicone is suitably 3:1, particularly 2:1, and more particularly 1:1.

Although the liquid components of compositions described herein are important as they provide plasticity and help to avoid a product that is too hard, brittle or flaky and thus uncomfortable, compositions that contain a high proportion of components that are liquid at room temperature are more difficult to process. Upon freezing, solid components, especially immobilizing agents, are important for providing a network that is capable of supporting the liquid components within it and, therefore, preventing their migration through the substrate. When the solid portion of the moisturizing and lubricating composition is too small, the formed network may be overwhelmed by the large liquid portion making the solids unable to support the liquids in the network, which can then result in substantial migration by the liquid portions into the matrix of the fabric of the product.

In order to avoid this potential problem of having too high of a proportion of liquids, compositions used with the present invention comprise no more than about 50% components that are liquid at room temperature, and no less than about 50% of components that are solid at room temperature. Compositions that are comprised of such components provide a favorable balance of liquid and solid components, and allow for easy processability while maintaining good aesthetic attributes.

As noted throughout herein, compatibility is important in providing a composition that is easily processable and stable. As such, the moisturizing and lubricating compositions described herein possess high compatibility and have at least about 85% (by weight), suitably at least about 90% (by weight), particularly at least about 94% (by weight), and more particularly at least about 97% (by weight) of the components in a single phase at a temperature of from about 45° C. to about 80° C. With such high compatibility, the compositions provide a significant advantage over prior compositions and are easily processable and stable. Additionally, it is preferable that the freezing point of the compositions be higher than room temperature to avoid the need for a cooling mechanism to process the compositions.

In addition to the emollient, humectant, immobilizing agent, and compatibilizing components described herein, compositions used with the present invention may optionally include a skin barrier enhancing agent, such as a fat or oil (triglyceride/essential and non-essential fatty acid containing), to enhance the barrier function of the stratum corneum layer of the skin or mucous membrane. Skin and mucous membranes are comprised mainly of cholesterol, ceramides, and fatty acids. The addition of fats, oils, or triglycerides alone or in combination with sterols or sterol derivatives to the composition can refat/replenish or enhance the natural lipid barrier of the skin or mucous membranes that comes in contact with the appliance comprising the composition. In addition to reforming structures needed to provide the proper lipid structures in the skin, the natural fats and oils will not substantially negatively affect the natural skin barrier if repair is not necessary.

The skin barrier enhancing agent is typically from about 0.1% (by weight) to about 30% (by weight), suitably from about 0.5% (by weight) to about 20% (by weight), and particularly from about 1% (by weight) to about 10% (by weight). Suitable fats or oils, or mixtures thereof, for inclusion in the moisturizing and lubricating compositions of the present invention typically comprise glycerides, triglycerides and/or essential and/or non-essential fatty acids. Suitable examples include apricot kernel oil, avocado oil, babassu oil, borage seed oil, butter, C₁₂-C₁₈ acid triglyceride, camellia oil, canola oil, caprylic/capric/lauric triglyceride, caprylic/capric/linoleic triglyceride, caprylic/capric/stearic triglyceride, caprylic/capric triglyceride, carrot oil, cashew nut oil, castor oil, cherry pit oil, chia oil, cocoa butter, coconut oil, cod liver oil, corn germ oil, corn oil, cottonseed oil, C₁₀-C₁₈ triglycerides, egg oil, epoxidized soybean oil, evening primrose oil, glyceryl triacetyl hydroxystearate, glyceryl triacetyl ricinoleate, glycosphingolipids, grape seed oil, hazelnut oil, human placental lipids, hybrid safflower oil, hybrid sunflower seed oil, hydrogenated castor oil, hydrogenated castor oil laurate, hydrogenated coconut oil, hydrogenated cottonseed oil, hydrogenated C₁₂-C₁₈ triglycerides, hydrogenated fish oil, hydrogenated lard, hydrogenated menhaden oil, hydrogenated mink oil, hydrogenated orange roughy oil, hydrogenated palm kernel oil, hydrogenated palm oil, hydrogenated peanut oil, hydrogenated shark liver oil, hydrogenated soybean oil, hydrogenated tallow, hydrogenated vegetable oil, lard, lauric/palmitic/oleic triglyceride, lesquerella oil, linseed oil, macadamia nut oil, maleated soybean oil, meadowfoam seed oil, menhaden oil, mink oil, moringa oil, mortierella oil, neatsfoot oil, oleic/linoleic triglyceride, oleic/palmitic/lauric/myristic/linoleic triglyceride, oleostearine, olive husk oil, olive oil, omental lipids, orange roughy oil, palm kernel oil, palm oil, peach kernet oil, peanut oil, pengawar djambi oil, pentadesma butter, phospholipids, pistachio nut oil, placental lipids, rapeseed oil, rice bran oil, safflower oil, sesame oil, shark liver oil, shea butter, soybean oil, sphingolipids, sunflower seed oil, sweet almond oil, tall oil, tallow, tribehenin, tricaprin, tricaprylin, triheptanoin, trihydroxymethoxystearin, trihydroxystearin, triisononanoin, triisostearin, trilaurin, trilinolein, trilinolenin, trimyristin, trioctanoin, triolein, tripalmitin, trisebacin, tristearin, triundecanoin, vegetable oil, walnut oil, wheat bran lipids, wheat germ oil, and zadoary oil.

When compositions used with the present invention comprise a skin enhancing agent such as a fat or oil described above, the composition generally will also comprise an antioxidant. Compositions suitable for use on absorbent and tissue products that contain a fat or oil skin enhancing agent without an antioxidant tend to develop an offensive odor making the product commercially unsuitable. This problem is especially acute when the products are exposed to elevated temperatures during processing, storage and shipping. It has been discovered that the offensive odor is attributable to the partial or complete oxidation of the fat or oil. As such, by introducing an antioxidant into the moisturizing and lubricating compositions of the present-invention, the development of an offensive odor over time can be substantially minimized or eliminated resulting in a substantially improved commercial product.

In addition to minimizing or eliminating offensive odors which can be produced by natural fats or oils, the antioxidant may provide a skin health benefit by repairing damaged lipids on the skin's surface. The epidermal lipids consist of a high proportion of polyunsaturated fatty acids, which are susceptible to oxidation on the skin's surface. Oxidation of these polyunsaturated fatty acids can be initiated by a number of means including metals, such as iron, which are naturally present in the skin as well as in menstrual fluid. Antioxidants can prevent or repair oxidative damage to polyunsaturated fatty acids as well as other oxidation prone constituents in the skin.

The antioxidant is present in compositions used in the present invention in an amount of from about 0.05% (by weight) to about 5% (by weight), suitably from about 0.1% (by weight) to about 2% (by weight), and particularly from about 0.1% (by weight) to about 1% (by weight). Antioxidants suitable for reducing the likelihood of the fats and/or oils to oxidize and produce an unwanted odor include natural and synthetic tocopherol, butylated hydroxyanisole (BHA), butylated hydroxytoluene (BHT), carotenoids, filtered wheat germ oil, gamma oryzanol, sodium sulfite, grape seed extract, green tea extract, rosmaric acid, ubiquinone, lipoic acid, N-acetyl-cysteine, avocado, sage, and proanthrocyanidins. Particularly preferred antioxidants include natural and synthetic tocopherol, BHT, and gamma oryzanol. Synthetic tocopherols include, for example tocopherol acetate, tocopherol linoleate, tocopherol succinate, tocopherol sorbate, tocotrienol, and Trolox (6-hydroxy-2,5,7,8-tetramethyl chromane-2carboxylic acid).

Compositions used with the present invention may also optionally include a sterol or sterol derivative or mixture of sterols and sterol derivatives to provide a skin health benefit. Typically, compositions may include from about 0.1% (by weight) to about 10% (by weight), suitably from about 0.5% (by weight) to about 5% (by weight) and particularly about 1% (by weight) of sterol or sterol derivative. Suitable sterols and sterol derivatives for incorporation into the compositions used with the present invention include, for example, cholesterol sulfate, beta-sterols having a tail on the 17 position and having no polar groups, for example cholesterol, sitosterol, stigmasterol, and ergosterol, as well as C₁₀-C₃₀ cholesterol/lanosterol esters, cholecalciferol, cholesteryl hydroxystearate, cholesteryl isostearate, cholesteryl stearate, 7-dihydrocholesterol, dihydrocholesterol, dihydrocholesteryl octyidecanoate, dihydrolanosterol, dihydrolanosteryl octyldecanoate, ergocalciferol, tall oil sterol, soy sterol acetate, lanasterol, soy sterol, avocado sterols, cholesterol esters, sterol esters, and the like, as well as mixtures thereof.

Compositions used with the present invention may also optionally include other components such as emulsifiers, surfactants, water, viscosity modifiers, pH modifiers, enzyme inhibitors/inactivators, suspending agents, pigments, dyes, ceramides such as glucosylceramides, colorants, buffers, perfumes, antibacterial actives, antifungal actives, pharmaceutical actives, film formers, deodorants, opacifiers, astringents, solvents, organic acids, coloring agents, preservatives, antivirul actives, drugs, vitamins, aloe vera, panthenol, natural moisturizing factors, and the like. These materials are known in the art and are used in their art-established manner at their art-established amounts.

The compositions of the present invention are introduced onto the desired product in an amount sufficient to provide a benefit, e.g., a moisturizing and/or lubricating benefit. For example, compositions of the present invention may be introduced onto a body-facing surface of an appliance in an amount of from about 0.05 g/m² to about 100 g/m², suitably from about 1.0 g/m² to about 40 g/m², and particularly from about 4 g/m² to about 15 g/m².

The coating may be deposited on the interior surface of the appliances by any suitable method. For example, the appliances may be dipped in the coating (and then inverted—this assumes that the appliance was first turned inside out to expose the inner surface of the appliance before treatment with a formulation). In an alternative embodiment, the appliances may be tumbled in the coating. In various embodiments, the coating may be applied to the surface of the appliance through dipping, immersion, spraying, patting, printing, brushing, or any other application method known in the art.

In one embodiment, the coating may be sprayed onto a skin-contacting surface of the appliance. For instance, appliances may be placed in a tumbling apparatus while a solution of the coating is sprayed on the gloves. In one embodiment, the spraying process may be repeated. For instance, the spraying process may be repeated up to about twenty times to coat the inner surface of the gloves. In one embodiment, the spraying process may be carried out for a total of between about ten and about twenty times.

Whatever the method of application, the formulation is heated so that the formulation may be processed (e.g., may be pumped and/or directed through piping and/or directed through slot-coating equipment, spray nozzles, and the like; etc.), which generally means that the formulation is in liquid form immediately prior to its application to the liquid-permeable substrate. After application to the liquid-permeable substrate, the formulation will “freeze”—i.e., go from the liquid-phase to a solid or semi-solid phase—as the temperature of the formulation drops. Once the formulation is a solid or semi-solid it is substantially immobile (i.e., will not migrate through the substrate during storage, shipment, or use such that the formulation reaches an exterior surface of the appliance in significant quantities, thereby contaminating any surface that a user of the appliance contacts while wearing the appliance). Because the formulation needs to be heated so that it may be processed, but not so much that the formulation fails to “freeze” shortly after it's application to the substrate, thereby potentially allowing the formulation—while in liquid form—to migrate entirely through the substrate so that it is associated with both surfaces of the substrate, only that amount of energy needed for processing purposes should be inputted into the formulation (e.g., by heating)). Of course if the formulation is applied to one layer or composite of the appliance, and then allowed to freeze, with the impregnated layer or composite then attached to one or more other layers making up a multi-layer appliance, then the formulation might be allowed to completely penetrate through the layer or composite to which it is applied.

### Representative Marketing and/or Packaging of Appliances of Present Invention

The manufacturer of an appliance of the present invention may fashion messages, statements, or copy to be transmitted to a purchaser, consumer, or user of said appliance. Such messages, statements, or copy may be fashioned to help facilitate or establish an association in the mind of a person/customer/shopper/user between an appliance of the present invention, or use thereof, and one or more mental states, psychological states, or states of well being. The communication, statements, or copy may include various alphanumeric strings, including, for example: “moisture”, “moisturize”, “moisturizing”, “pamper”, “pampering”, “ritual”, “personal”, “spa”, “treatment”, “foot”, “hand”, “system”, “effective”, “convenient”, “disposable”, “botanical”, “vitamin”, “relax”, “peace”, “energy”, “energize”, “sex”, “sensuality”, “sensual”, “spirit”, “spiritual”, “clean”, “fresh”, “mountain”, “country”, “zest”, “sea”, “sky”, “health”, “hygiene”, “water”, “waterfall”, “moisture”, “moisturize”, or derivatives or combinations thereof. It should be noted that each term appearing in quotes in the preceding list may be in any font, style, color, etc.—and the quotes likely would not appear around the term when the term is employed. These alphanumeric strings may be used either alone, adjacent to, or in combination with, other alphanumeric strings. In one embodiment, the communication, statements, or copy associate an appliance of the present invention and a registered or common-law trademark, name, brand name, and/or logo of the seller or manufacturer of the appliance (and/or health-and-beauty products generally).

The communication, statements, message, or copy could take the form of (i.e., be embodied in a medium such as) a newspaper advertisement, a television advertisement, a radio or other audio advertisement, items mailed directly to addressees, items emailed to addresses, Internet Web pages or other such postings, free standing inserts, coupons, various promotions (e.g., trade promotions), co-promotions with other companies, copy and the like, boxes and packages containing the product (in this case an appliance of the present invention), and other such forms of disseminating information to consumers and/or customers or potential consumers and/or customers.

It should be noted that when associating statements, copy, messages, or other communications with a package (e.g., by printing text, images, symbols, graphics, color(s), or the like on the package; or by placing printed instructions in the package; or by associating or attaching such instructions, a coupon, or other materials to the package; or the like) containing appliances of the present invention, the materials of construction of said package may be selected to reduce, impede, or eliminate the passage of water or water vapor through at least a portion of the package. The materials of construction may also be selected to reduce, impede, or eliminate the passage of ultraviolet light (or other electromagnetic radiation) through at least a portion of the package. Furthermore, appliances may be individually wrapped in containers, packets, envelopes, bags, or the like that inhibit, reduce, or eliminate the passage or transmission of water or water vapor from appliances contained therein. For purposes of this application, “packages,” “containers,” “envelopes,” “bags,” “packets,” and the like are interchangeable in the sense that they refer to any material adapted to enclose and hold either individual appliances (as in, for example, an individual packet containing a single appliance), or a plurality of appliances (as in a flexible bag made of film containing a plurality of appliances, whether or not each of the individual appliances are enclosed and held in a separate material—such as individual packets).

Appliances of the present invention may be sold in packages containing other personal-care articles, such as a buff (e.g., an exfoliating buff); a body, facial, or other cloth (e.g., a clothing comprising nonwoven materials and/or film materials and/or elastomeric materials and/or formulation(s)); a pouf; formulations; and the like.

These and other modifications and variations to the present invention may be practiced by those of ordinary skill in the art, without departing from the spirit and scope of the present invention, which is more particularly set forth in the appended claims. Furthermore, those of ordinary skill in the art will appreciate that the foregoing description is by way of example only, and is not intended to limit the invention so further described in such appended claims.

### EXAMPLE 1

### Representative Examples of Compositions Used with the Present Invention

Exemplary personal-care compositions were prepared having the ingredients/components and proportions identified below in Tables 1, 2, and 3:

TABLE 1

| | % by |
|---|---|
| Name | Weight |
| Petrolatum | 75 |
| Stearyl benehate | 15 |
| C26–34 Olefin | 6 |
| AC400 | 4 |

Petrolatum is heated to 85° C. Stearyl behenate, C26-34 Olefin and AC400 are added to the molten petrolatum and the mixture is maintained at 85° C. until all ingredients are molten. The final composition is then allowed to cool while mixing until it changes to a semisolid state. At this point the mixing is stopped and the composition is allowed to cool further until it equilibrates to room temperature.

TABLE 2

| | % by |
|---|---|
| Name | Weight |
| Table 1 composition | 67.6 |
| C24–28 Alkyl Methicone | 15.0 |
| Dimethicone 200 | 15.0 |
| Dimethicone and Dimethiconol | 2.0 |
| Fragrance | 0.2 |
| Polysorbate 80 | 0.2 |

The composition in Table 1 is prepared as described above. However, before that composition is allowed to cool, the C24-28 alkyl methicone, dimethicone, and dimethicone/dimethiconol are added to the molten composition from Table 1 and the mixture is maintained at 85° C. until all ingredients are molten. The composition is then allowed to cool while mixing until it changes to a semisolid state. Separately, the fragrance is mixed with polysorbate 80. The fragrance/polysorbate 80 mixture is then added to the semisolid mixture and mixed well to form the final formulation. The mixing is then is stopped and the composition is allowed to cool further until it equilibrates to room temperature.

TABLE 3

| | % by |
|---|---|
| Name | Weight |
| Glycerin | 10 |
| PEG-12 | 15 |
| PEG-20 | 20 |
| PEG-75 | 25 |
| PEG-150 | 25 |
| Dimethicone | 2.5 |
| Dimethicone and Dimethiconol | 2.5 |

The PEG-150 is melted by heating to 80° C. The PEG-75, PEG-20, and PEG-12 are added to the molten PEG-150 in this order. Each of these ingredients is added after the previous ingredient melts fully. Glycerin is then added, followed by dimethicone and dimethicone/dimethiconol. The final composition is then allowed to cool while mixing until it changes to a semisolid state. At this point the mixing is stopped and the composition is allowed to cool further until it equilibrates to room temperature. Optionally, fragrance and polysorbate 80 can be added to the composition in a manner similar to that described under Table 2.

### EXAMPLE 2

### Prophetic; Representative Appliances

An exemplary prophetic appliance is prepared in the following manner.

A composite of spaced-apart, substantially parallel elastic strands attached to meltblown fiber is produced. First, elastic strands are extruded through orifices having an inside diameter of 0.030 inches, with a spacing of 10 orifices per inch across the width of the web being formed. The extruded strands are composed of Kraton 6638 polymer resin (Kraton 6638 is a blend of 80% by weight Kraton 1730 styrene-(ethylene-propylene)-styrene-(ethylene-propylene) tetrablock copolymer from Kraton Polymers LLC, 7% by weight PETROTHANE NA601 polyethylene wax from Quantum Chemical Co., and 13% by weight REGALREZ 1126 tackifier from Eastman Chemical Co).

After the elastic strands have been formed, meltblown fiber is attached to the elastic strands. The meltblown fiber is composed of a polypropylene grade elastomer, VistaMaxx 1100, available from ExxonMobil Chemical, a business having offices in Houston, Tex. The polymeric elastomer is heated to a temperature of 420 degrees Fahrenheit. The molten polymer is then directed though orifices having an inside diameter of 0.0145 inches, with 30 orifices per inch across the width of the web being formed. The bank of orifices is positioned such that there is a distance of 10 inches between the orifices and the web being formed. Air at a temperature of 420 degrees Fahrenheit is directed at the molten polymer strands being extruded through the orifices to attenuate the strand concurrent to the forming of a network of meltblown fiber attached to the elastic strands.

As elastic strand/meltblown composite is being produced (at a 75 grams per square meter basis weight prior to stretching), it is stretched by about 400% along the dimension parallel to the direction of the composite.

To both sides of the elastomeric composite, a necked polypropylene spunbond material, having a basis weight of 0.8 ounces per square yard, is attached. This is accomplished by first spraying a hot-melt adhesive, #2840 (available from Ato-Findley), at an add-on level of 2 grams per square meter, and heated to a temperature of 365 degrees Fahrenheit, to the side of the spunbond material that is to contact the elastomeric composite.

In summary, the elastomeric composite is sandwiched between two fibrous layers, with one fibrous layer on the other side of the elastomeric composite. Because neither fibrous layer is stretched when attached to the elastomeric composite, and because both fibrous layers are attached at discrete locations along the length of the elastomeric composite, both fibrous layers are gathered when the elastomeric composite is allowed to retract. A cross-section of the resulting substrate would look something like that depicted in FIG. 1C.

Once the substrate is prepared, it is cut into individual pieces in the shape of a hand and foot (similar to the shapes representatively depicted in FIGS. 2, 2A, 3, and 3A above). The individual pieces are then sewn together to make either a glove into which a user could insert his or her hand, or a sock into which a user could insert his or her foot.

A personal-care composition is then applied. Each of the formulations identified in Examples 1 above is added to different pairs of socks and gloves. The formulations are applied by heating the formulations so that they are molten and may be processed. In this case the formulations are sprayed onto the exposed surfaces of the appliances (with the seam visible), at an add-on amount of approximately 5 g per appliance side (for a total of approximately 10 grams of formulation for each appliance). The formulation subsequently freezes, and the product/appliance is then inverted back with the seams inside and placed in air-tight bags.

### EXAMPLE 3

### Prophetic; Representative Appliances

The prophetic, exemplary appliances of Example 2 are prepared, with the exception that: the fibrous layers of Example 2 are thermally bonded to the elastomeric composite without an adhesive. Methods disclosed elsewhere in the application, and known in the art of thermal bonding, are used to join the referenced materials to form the described appliances.

1. A disposable appliance adapted to contact the skin of a wearer of the appliance with a composition associated with an interior, body-facing surface of the appliance, the appliance comprising:
a first liquid-permeable substrate having a perimeter;
a second liquid-permeable substrate having a perimeter, wherein the first liquid-permeable substrate and the second liquid-permeable substrate are attached to one another in a way that defines an interior volume into which a wearer of the appliance may insert a portion of his or her body, said interior volume bounded in part by an interior, body-facing surface; and
a substantially immobile composition associated with at least a portion of the interior, body-facing surface.

2. The appliance of claim 1 wherein the first and second liquid-permeable substrates comprise a nonwoven.

3. The appliance of claim 1 wherein the first and second liquid-permeable substrates comprise an apertured film.

4. The appliance of claim 1 wherein the first and second liquid-permeable substrates comprise elastic strand.

5. The appliance of claim 1 wherein the substantially immobile composition is adapted to moisturize skin.

6-7. (canceled)

8. The appliance of claim 1 wherein the composition comprises a humectant.

9. The appliance of claim 1 wherein the composition comprises an emollient, a humectant, an immobilizing agent, and a compatibilizing agent.

10. The appliance of claim 1 wherein the first and second liquid-permeable substrate comprises a gathered nonwoven attached to an elastic layer.

11-15. (canceled)

16. A disposable appliance adapted to contact the skin of a wearer of the appliance with a composition associated with an interior, body-facing surface of the appliance, the appliance comprising:
a liquid-permeable substrate having a perimeter, wherein the substrate is attached to itself in a way that defines an interior volume into which a wearer of the appliance may insert a portion of his or her body, said interior volume bounded in part by an interior, body-facing surface;
a substantially immobile composition associated with at least a portion of the interior, body-facing surface.

17. The appliance of claim 16 wherein the liquid-permeable substrate comprises a nonwoven.

18. The appliance of claim 16 wherein the liquid-permeable substrate comprises an apertured film.

19. The appliance of claim 16 wherein the liquid-permeable substrate comprises elastic strand.

20. The appliance of claim 16 wherein the substantially immobile composition is adapted to moisturize skin.

21-22. (canceled)

23. The appliance of claim 16 wherein the composition comprises a humectant.

24. The appliance of claim 16 wherein the composition comprises an emollient a humectant, an immobilizing agent, and a compatibilizing agent.

25. The appliance of claim 16 wherein the liquid-permeable substrate comprises a gathered nonwoven attached to an elastic layer.

26. The appliance of claim 16 wherein the appliance is a glove, sock, or sleeve.

27. A package, the package comprising:
a plurality of appliances of claim 16, each contained in an envelope; and
a container for the plurality of appliance-containing envelopes.

28. The package of claim 27 wherein each envelope contains a pair of appliances.

29. The package of claim 27 further comprising a second personal-care article of manufacture.

30. The package of claim 27 further comprising a statement disposed in, on, or proximate to said container, wherein the statement associates the appliance with one or more of the following: “moisture”, “moisturize”, “moisturizing”, “pamper”, “pampering”, “ritual”, “personal”, “spa”, “treatment”, “foot”, “hand”, “system”, “effective”, “convenient”, “disposable”, “botanical”, “vitamin”, “relax”, “peace”, “energy”, “energize”, “sex”, “sensuality”, “sensual”, “spirit”, “spiritual”, “clean”, “fresh”, “mountain”, “country”, “zest”, “sea”, “sky”, “health”, “hygiene”, “water”, “waterfall”.

31. A message adapted to be communicated to consumers, wherein the message is based, in whole or in part, on information relating to an appliance of claim 1 or 16, and wherein the message is contained in text, a symbol, a graphic, an image, and/or color, and wherein the message is embodied in a medium capable of being transmitted to consumers.

32. A disposable appliance adapted to contact the skin of a wearer of the appliance with a composition associated with a body-facing surface of the appliance, the appliance comprising:
a liquid-permeable substrate having a perimeter and a body-facing surface; and
a substantially immobile composition associated with at least a portion of the interior, body-facing surface.

33. The appliance of claim 32 wherein the appliance is a patch adapted to releasably engage the skin of a user of the appliance.

34. The appliance of claim 33 wherein the appliance comprises an adhesive adapted to releasably engage the skin of a user of the appliance.
